(19) United States
(12) Patent Application Publication
(10) Pub. No.: US 2009/0036418 A1
(43) Pub. Date: Feb. 5, 2009

(54) Pharmaceutical composition for treatment of cancer containing pyridylpyridazine or transition metal complex thereof as active ingredient

(76) Inventor: Nack-Do Sung, Daejeon (KR)

Correspondence Address: STAAS & HALSEY LLP, SUITE 700, 1201 NEW YORK AVENUE, N.W., WASHINGTON, DC, 20005, US

(21) Appl. No.: 11/988,729

(22) PCT Filed: Feb. 7, 2006
(86) PCT No.: PCT/KR2006/000449

§ 371 Date: Jan. 14, 2008

(30) Foreign Application Priority Data
Jul. 14, 2005 (KR) 10-2005-0063710

(51) Int. Cl. A61K 31/501; A61K 31/555; A61P 35/00

(52) U.S. Cl. 514/188; 514/252.03

(57) ABSTRACT

The present invention relates to a pharmaceutical composition for the treatment of cancer, which contains, as an active ingredient, a pyridylpyridazine compound or a transition metal complex thereof.

As described above, the pyridylpyridazine compounds according to the present invention and the transition metal complexes thereof have anticancer activity and can be easily prepared at low cost. Thus, these compounds are useful as anticancer agents against various cancers, including lung cancer, adenocarcinoma, skin cancer, colon cancer, uterine cancer and brain cancer, which are expressed due to immune system abnormality.

### CROSS-REFERENCE TO RELATED APPLICATIONS

This application claims the benefit of Korean Application No. 10-2005-0063710, filed Jul. 14, 2005, in the Korean Intellectual Property Office, and the PCT Application No. PCT/KR2006/000449, filed Feb. 7, 2006, the disclosure of which is incorporated herein by reference.

### BACKGROUND

1. Field

The present invention relates to a pharmaceutical composition for the treatment of cancer, which contains, as an active ingredient, a pyridylpyridazine compound of Formula 1 or a transition metal complex thereof.

2. Description of the Related Art

With the development of modern medical science, many diseases have been treated, but cancer still remains as one of difficult-to-treat diseases. Cancer is the first leading cause of death in most of countries, including Korea, and has continued to increase.

Methods for the treatment of cancer include chemical therapy, immune therapy, surgical therapy and radiation therapy. Among these methods, the immune therapy is believed to be an excellent therapeutic method in theoretical terms or against the cancer of experimental animals, but has insufficient therapeutic effects, and the use thereof is limited only to some cancers. Thus, up to this point of time, the chemical therapy has been used as the most important therapy in cancer therapy, particularly systemic therapy. These days, about 60 kinds of various anticancer drugs are used in clinical trials. Also, as much knowledge on cancer incidence and the characteristics of cancer cells is known, new anticancer drugs have been continuously developed.

Among anticancer drugs developed to date, cis-platin, an useful drug, is a platinum complex anticancer agent, which has a platinum (Pt) atom in the center of the molecular structure and is adhered to a DNA double strand structure present in the nucleus of cancer cells, so that it exhibits antitumor activity (anticancer effect) of suppressing DNA replication to inhibit cancer cell growth and proliferation and removing cancer cells. Cis-platin is widely used for the treatment of testicular cancer, ovarian cancer, lung cancer, head and neck cancer, bladder cancer, stomach cancer and cervical cancer, but has problems in that it causes side effects, including hematopoietic toxicity such as anemia, digestive toxicity such as vomiting and nausea, nephrotoxicity, and neuronal toxicity (R. T. Skeel, Handbook of Cancer Chemotherapy, pp 89-91, 1999), and loses anticancer activity due to resistance acquisition of cancer cells. For this reason, carboplatin, a second-generation platinum complex anticancer agent, was developed. Carboplatin showed a great reduction in nausea, vomiting and nephrotoxicity, which are the main toxicities of cis-platin, but it has problems in that it has strong marrow toxicity, low anticancer activity lower and a narrow spectrum of anticancer action, compared to those of cis-platin. For this reason, the development of anticancer drugs based on a third-generation platinum complex compound, which shows potent anticancer activity while having reduced side effects and a broad spectrum of anticancer action, compared to those of cis-platin, is actively ongoing in Korea and foreign countries.

Meanwhile, regarding transition metal complexes other than platinum, Korean Patent No. 10-378257 discloses a biscarboxyethylgermanium sesquioxide-transition element complex, which contains, as a ligand, biscarboxyethylgermanium sesquioxide known as an anticancer agent in the prior art. However, there are almost no studies on the possibility of use thereof as an anticancer agent.

Accordingly, considering that transition metal complexes other than platinum complexes can be easily prepared at low cost compared to the platinum complexes, the present inventors have investigated the anticancer activity of the transition metal complexes in the human body and, as a result, found that a pyridylpyridazine compound of Formula 1 and a transition metal complex having said compound as a ligand exhibits high anticancer activity against a very broad spectrum of human cancer cell lines, thereby completing the present invention.

### SUMMARY

Additional aspects and/or advantages will be set forth in part in the description which follows and, in part, will be apparent from the description, or may be learned by practice of the invention.

To achieve the above object, the present invention provides a pharmaceutical composition containing pyridylpyridazine compound of Formula 1 as an active ingredient:

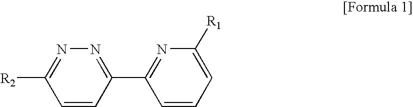

wherein R₁ is hydrogen or C₁—C₅ lower alkyl, R₂ is hydrogen or

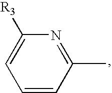

and R₃ is hydrogen or C₁—C₅ lower alkyl.

Also, the present invention provides a pharmaceutical composition containing a transition metal complex of the pyridylpyridazine compound of Formula 1 as an active ingredient.

Hereinafter, the present invention will be described in further detail.

The pyridylpyridazine compound of Formula 1 according to the present invention and the transition metal complex having the compound as a ligand will be described in further detail with reference to preparation methods thereof.

The pyridylpyridazine compound of Formula 1 according to the present invention can be readily synthesized on the basis of technology known in the literature[Butte, W. A., Case, F. H., The synthesis of some pyridylpyridazines and and pyrim-idines, J. Org. Chem. 26, 4690-4692 (1961); Heldmann, D. K.; Sauer, J. Synthesis of metallated (metal=Si, Ge, Sn) pyridazine by cycloaddition of metal substituted alkynes to 1,2,4,5-tetrazine; Tetrahedron Letter. 38, 5791-5794, (1997)].

Preferably, a compound in which R₂ in Formula 1 is hydrogen can be prepared according to an organic synthetic method as shown in Reaction Scheme 1:

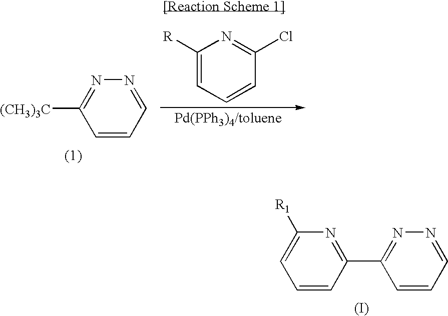

As shown in Reaction Scheme 1, a solution of pyridazine in an organic solvent such as ether or pentane is allowed to react with butyllithium, and the reaction product is hydrolyzed and then extracted with ether, thus obtaining butylpyridazine (1). The butylpyridazine is allowed to react with bromopyridine in anhydrous toluene in the presence of a palladium catalyst, thus preparing the pyridylpyridazine compound of Formula 1 according to the present invention.

Also, a bispyridylpyridazine compound in which R₂ is

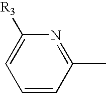

can be prepared according to an organic synthetic method as shown in Reaction Scheme 2:

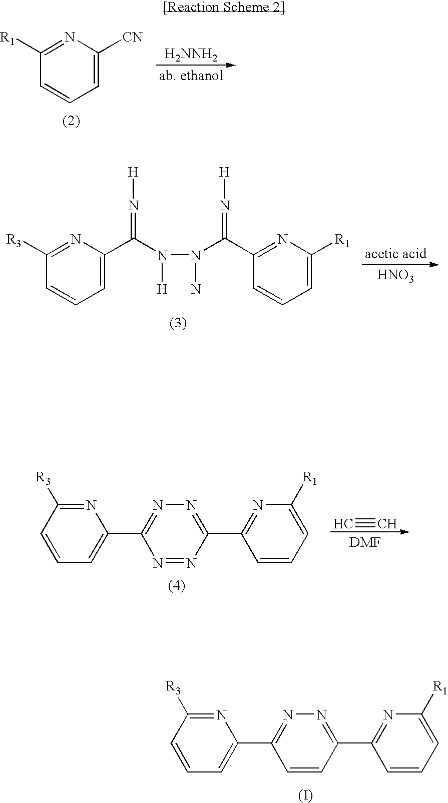

As shown in Reaction Scheme 2, a cyanopyridine compound (2) is allowed to react with a hydrazine compound in anhydrous ethanol so as to obtain an anhydrous base (3), which is then allowed to react with nitric acid in a mixed solvent of hydrogen peroxide and glacial acetic acid, thus obtaining a 3,6-bis(2′-pyridyl)-1,2,4,5-tetrazine compound (4). The compound (4) is allowed to react with acetylene in a solvent such as DMF, thus obtaining the pyridylpyridazine compound (1) of Formula 1 according to the present invention.

The pyridylpyridazine compound (1) of Formula 1 according to the present invention is preferably a compound in which R₁ is hydrogen or methyl, and R₂ is hydrogen, pyridine or methylpyridine. More preferably, the pyridylpyridazine compound (1) represented by Formula 1 is 3-(pyridine-2-yl)pyridazine (hereinafter, referred to as 2′-pyridylpyridazine), 3-(6-methylpyridine-2-yl)pyridazine (hereinafter, referred to as 6′-methyl-2′-pyridylpyridazine), 3,6-bis(2′-pyridyl)pyridazine, or 3,6-bis(6′-methyl-2′-pyridyl)pyridazine. Most preferably, it is 3,6-bis(2′-pyridyl)pyridazine in which R₁ is hydrogen and R₂ is pyridine.

The transition metal complex of the pyridylpyridazine compound (1) of Formula 1 according to the present invention can be readily synthesized according to a method known in the literature[Sung, N. D. et al, μ-Aqua-pentaaqua[μ-3,6-bis(6′-methyl-2-pyridyl)pyridazine]chlorodinickel(II)trichloride trihydrate. Acta. Cryst. C56, e370-e371. 2000; Sung, N. D. et al, Bis [3,6-bis(6′-methyl-2′-pyridyl)-pyridazine-k²N²,N³]chlorocopper (II) perchlorate. Acta. Cryst. C57, 47-48 (2001); Kim, M. J. et al., The crystal structure of [3,6-bis(6′-methyl-2′-pyridyl)pyridazine]ZnCl₂, C₁₆H₁₆N₄, ZnCl₂, Kor. J. Crystallography. 10, 119-124 (1999)], and can be prepared according to an organic synthetic method as shown in Reaction Scheme 3:

[Reaction Scheme 3]

(I)+MX₂→(I)−MX₂+(I)₂−MX₂

wherein MX₂ denotes zinc chloride, copper chloride or nickel chloride for the preparation of the transition metal complex, M denotes a transition metal, and X denotes halide. Halide X reacts with water to form a water molecule or a hydroxyl group.

More specifically, the transition metal complex of the pyridylpyridazine compound (1) of Formula 1 can be prepared according to an organic synthetic method as follows. A solution of one equivalent of the pyridylpyridazine compound (1) in acetone is added to a solution of 1-2 equivalents of a metal compound in distilled water. Then, one equivalent of sodium perchlorate is added and allowed to react with the solution mixture, and the reaction product is crystallized in a cold dark place, thus obtaining the transition metal complex of the pyridylpyridazine compound (1) of Formula 1.

The transition metal complex of the pyridylpyridazine compound (1) of Formula 1 according to the present invention is prepared using the pyridylpyridazine compound (1) as a ligand. Herein, the transition metal is selected from the group consisting of nickel, copper and zinc.

In synthesizing a transition metal complex using the pyridylpyridazine compound (1) as a ligand, each of complex compounds such as ligand monomers (zinc and nickel) and ligand dimers (copper) can be synthesized by changing the ratio of ligand compound to metal compound added. The structural formula of each of the transition metal complexes is shown in Preparation Examples of the compounds.

The transition metal complex of the pyridylpyridazine compound (1) of Formula 1 according to the present invention is preferably a nickel, copper or zinc complex compound, which has 2′-pyridylpyridazine, 6′-methyl-2′-pyridylpyridazine, 3,6-bis(6′-methyl-2′-pyridyl)pyridazine or 3,6-bis(2′-pyridyl)pyridazine as a ligand. More preferably, it is a [2′-pyridylpyridazine]zinc(II) complex, a [6′-methyl-2′-pyridylpyridazine]zinc(II) complex, a [3,6-bis(2′-pyridyl)pyridazine]zinc(II) complex, a [3,6-bis(6′-methyl-2′-pyridyl)pyridazine]zinc(l I) complex, a bis[2′-pyridylpyridazine]copper(II) complex, a bis[6′-methyl-2′-pyridylpyridazine]copper(II) complex, a bis[3,6-bis(2′-pyridyl)pyridazine]copper(II) complex, a bis[3,6-bis(6′-methyl-2′-pyridyl)pyridazine]copper(II) complex, a [2′-pyridylpyridazine]nickel(II) complex, a [6′-methyl-2′-pyridylpyridazine]nickel(II) complex, a [3,6-bis(2′-pyridyl)pyridazine]nickel(II) complex or a [3,6-bis(6′-methyl-2′-pyridyl)pyridazine]nickel(II) complex. Still more preferably, it is a copper or zinc complex compound having 3,6-bis(6′-methyl-2′-pyridyl)pyridazine as a ligand. Most preferably, it is bis[3,6-bis(6′-methyl-2′-pyridyl)pyridazine]chlorocopper(II).

Also, the pyridylpyridazine compounds of Formula 1 according to the present invention or the transition metal complexes thereof may include derivative compounds in the form of pharmaceutically acceptable salts, hydrates or solvates.

The anticancer activity of the pyridylpyridazine compound of Formula 1 or the transition metal complex thereof was examined by screening 17 kinds of cancer cell lines from 60 kinds of cancer cell lines[Ross, D. T. et al., Systematic variation in gene expression patterns I human cancer cell lines. Nature genetics, 24, 227-235 (2000)], which are used for search of anticancer activity in the US National Cancer Institute, and evaluating the ED₅₀ value of the compound for the screened cell lines. As a result, the pyridylpyridazine compound of Formula 1 or the transition metal complex thereof showed anticancer activity against various human cancer cell lines, including lung cancer, skin cancer, colon cancer, uterine cancer and brain cancer cell lines (see Table 1). Among these compounds, the 3,6-bis(2′-pyridyl)pyridazine compound showed potent anticancer activity against various human caner cell lines, including lung cancer, skin cancer, colon cancer, uterine cancer and brain cancer cell lines, and particularly it showed substantially the same level of anticancer activity as that of the known anticancer agent cis-platin with respect to the brain cancer cell line (see Table 1).

Also, the copper complex of the pyridylpyridazine compound of Formula 1 showed substantially the same level of excellent anticancer activity as that of cis-platin with respect to various human cancer cell lines. Particularly, it showed excellent anticancer activity compared to that of cis-platin against adenocarcinoma, skin cancer, brain cancer and colon cell lines (see Table 2).

Accordingly, the pyridylpyridazine compound of Formula 1 or the transition metal complex thereof can be effectively used for the treatment of human cancer. Examples of cancers, to which the inventive compound can be applied, include, but are not limited to, lung cancer, adenocarcinoma, skin cancer, colon cancer, uterine cancer and brain cancer.

A pharmaceutical composition containing the pyridylpyridazine compound of Formula 1 or the transition metal complex thereof may comprise a pharmaceutically effective amount of the pyridylpyridazine compound of Formula 1 or the transition metal complex thereof alone or together with at least one pharmaceutically acceptable carrier, excipient or diluent. As used herein, the term “pharmaceutically acceptable amount” refers to an amount of the compound sufficient for treating cancer.

The pharmaceutically effective amount of the pyridylpyridazine compound of Formula 1 or the transition metal complex thereof is 0.1 μg/day/kg bodyweight to 10 mg/day/kg bodyweight, and preferably 0.1 μg/day/kg bodyweight to 0.1 mg/day/kg bodyweight. However, said pharmaceutically effective amount may be suitably varied depending on disease and its severity, the age, bodyweight, medical condition and sex of a patient, an administration route and treatment period.

The inventive compound can be used alone or in combination with other therapeutic methods (e.g., radiation therapy or surgical operation). Also, it can also be used in combination with other anticancer agents, for example, alkylating agents (e.g., cisplatin, carboplatin, etc.), metabolism agonists (e.g., methotrexate, 5-FU, etc.), anticancer antibiotics (e.g., adriamycin, bleomycin, etc.), anticancer plant alkaloids (e.g., taxol, etoposide, etc.), anticancer hormonal agents (e.g., dexamethasone, tamoxifen, etc.), anticancer drugs for the immune system (e.g., interferon α, β, γ, etc.).

As used herein, the term “pharmaceutically acceptable” refers to a composition which is physiologically acceptable and, when administered to the human beings, does not cause allergic reactions such as gastrointestinal disorders, or similar responses. Examples of said carrier, excipient or diluent may include lactose, dextrose, sucrose, sorbitol, mannitol, xylitol, erythritol, maltitol, starch, acacia rubber, alginate, gelatin, calcium phosphate, calcium silicate, cellulose, methyl cellulose, polyvinylpyrrolidone, water, methylhydroxybenzoate, propylhydroxybenzoate, talc, magnesium stearate, and mineral oil.

Said pharmaceutical composition may additionally comprise fillers, anticoagulants, lubricants, wetting agents, fragrances, emulsifiers and preservatives. Also, the inventive pharmaceutical composition can be formulated using a method known in the art so as to provide quick, sustained or delayed release of the active ingredient after administration to mammals. The composition may be in the form of powder, granules, tablets, emulsion, syrup, aerosol, soft or hard gelatin capsules, sterilized injection solution, or sterilized powder.

The composition according to the present invention can be administered through various routes, including oral, transdermal, subcutaneous, intravenous and intramuscular routes. The dosage of the active ingredient can be suitably selected depending on various factors, including an administration route and the age, sex, bodyweight and disease severity of a patient.

[Advantageous Effects]

As described above, the pyridylpyridazine compound of Formula 1 according to the present invention or the transition metal complex thereof has anticancer activity and can be easily prepared at low cost, and thus is useful for the treatment of various cancers, including lung cancer, adenocarcinoma, skin cancer, colon cancer, uterine cancer and brain cancer.

### DETAILED DESCRIPTION OF THE EMBODIMENTS

Reference will now be made in detail to the embodiments, examples of which are illustrated in the accompanying drawings, wherein like reference numerals refer to the like elements throughout. The embodiments are described below to explain the present invention by referring to the figures.

Hereinafter, the present invention will be described in further detail with reference to examples, but these examples are not to be construed to limit the scope of the present invention.

### PREPARATION EXAMPLE 1

### Preparation of 3,6-bis(2′-pyridyl)pyridazine

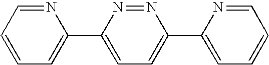

8.0 g (0.077 mole) of 2-cyanopyridine was dissolved in 200 ml of anhydrous ethanol, the solution was added into a three-neck flask, and 9.54 ml (0.308 mole) of 95% hydrazine hydrate was then added thereto. The mixture was refluxed for 6 hours to obtain 7.54 g (81.7% yield) of orange-colored dianhydride base. The base was filtered, washed several times with ethanol, dissolved in glacial acetic acid and cooled on an ice bath, and 5-6 ml of concentrated nitric acid was slowly added dropwise thereto. The mixture was poured over crushed ice, made basic with sodium bicarbonate and then extracted two times with chloroform. The organic layer was dried over anhydrous sodium sulfate and concentrated under reduced pressure, thus obtaining 6.3 g (85% yield) of 3,6-bis(2′-pyridyl)-1,2,4,5-tetrazine as a purple solid. 6.3 g (0.027 mole) of the obtained 3,6-bis(2′-pyridyl)-1,2,4,5-tetrazine was dissolved in dimethylformamide (hereinafter, referred to as DMF), and the solution was added into a three-neck flask and refluxed with bubbling of acetylene. As the reaction progressed, the mixture was changed from a purple color to a clear color. DMF was evaporated under reduced pressure, and the residue was extracted with ethyl acetate. The resulting solid material was purified by silica gel column chromatography using ethylacetate: n-hexane (1:4, v/v), thus obtaining 4.93 g (79% yield) of 3,6-bis(2′-pyridyl)_pyridazine

### PREPARATION EXAMPLE 2

### Preparation of 3,6-bis(6′-methyl-2′-pyridyl)pyridazine

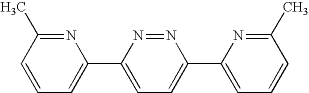

5.0 g (0.042 mole) of 2-cyano-6-methylpyridine was dissolved in 200 ml of anhydrous ethanol, the solution was added into a three-neck flask, and 12.33 ml (0.252 mole) of 95% hydrazine hydrate was added thereto. The mixture was refluxed for 6 hours, thus obtaining 2.86 g (50.5% yield) of a yellow dianhydride base. The base was filtered, washed several times with ethanol, dissolved in glacial acetic acid and cooled on an ice bath, and concentrated nitric acid was slowly added dropwise thereto. After completion of the addition, the mixture was stirred for 1 hour, poured on crushed ice, made basic with sodium bicarbonate and then extracted with chloroform, thus obtaining 2.67 g (78% yield) of purple-color 3,6-bis(6′-methyl-2′-pyridyl)-1,2,4,5-tetrazine.

2.67 g (0.01 mole) of the obtained 3,6-bis(6′-methyl-2′-pyridyl)-1,2,4,5-tetrazine was dissolved in DMF, and the solution was added into a three-neck flask and bubbled with acetylene. As the reaction progressed, the mixture was changed from a purple color to a clear color. DMF was evaporated under reduced pressure, and the residue was extracted with ethyl acetate and purified, thus obtaining 1.66 g (61% yield) of white 3,6-bis(6′-methyl-2′-pyridyl)pyridazine.

### PREPARATION EXAMPLE 3

### Preparation of bis[3,6-bis(6-methyl-2-pyridyl)pyridazine-k²N²,N³]copper(II) complex

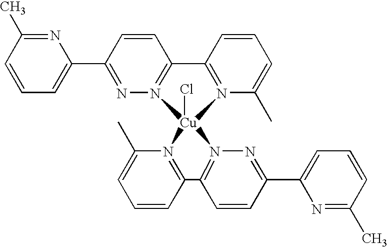

In 20 ml of acetone, 0.1 g (2 eq.) of the 3,6-bis(6′-methyl-2′-pyridyl)pyridazine compound prepared in Preparation Example 2 was allowed to react with 0.25 g (1 eq.) of copper chloride hexahydrate (CuCl₂.6H₂O), and 0.12 g (1 eq.) of NaClO₄ was then added thereto. The mixture was freeze-stored for one week, thus obtaining 0.15 g (85% yield) of bis[3,6-bis(6-methyl-2-pyridyl)pyridazine-k²N²,N³]chlorocopper(II) perchlorate as a green crystal. The complex was washed with anhydrous ethanol and dried at room temperature.

### PREPARATION EXAMPLE 4

### Preparation of [3,6-bis(6′-methyl-2′-pyridyl)pyridazine]zinc(II) complex

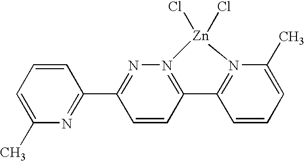

In 20 ml of anhydrous ethanol, 0.1 g (0.4 mM, 1 eq.) of the 3,6-bis(6′-methyl-2′-pyridyl)pyridazine compound prepared in Preparation Example 2 was allowed to react with 0.104 g (2 eq.) of zinc chloride (ZnCl₂), thus obtaining 0.116 g (58% yield) of white [3,6-bis(6′-methyl-2′-pyridyl)pyridazine]zinc(II) chloride.

### PREPARATION EXAMPLE 5

### Preparation of [3,6-bis(6′-methyl-2′pyridyl)pyridazine]nickel(II) complex

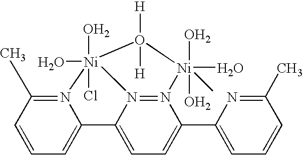

In 20 ml of anhydrous ethanol, 0.1 9 (0.38 mM, 1 eq.) of the 3,6-bis(6′-methyl-2′-pyridyl)pyridazine compound prepared in Preparation Example 2 was allowed to react with 0.18 g (2 eq.) of nickel chloride hexahydrate (NiCl₂.6H₂O), thus obtaining 0.16 g (61% yield) of μ-aqua-pentaaqua[μ-3,6-bis(6-methyl-2-pyridyl)pyridazine]chlorodinickel(II) trichloride trihydrate.

### PREPARATION EXAMPLE 6

### Preparation of 2′-pyridylpyridazine

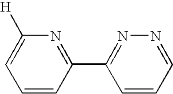

At a temperature of −15° C., a solution of 2 g of pyridazine in 120 ml of ether or pentane was slowly added dropwise to 60 ml of 0.5 M t-butyllithium with stirring over 1 hour and then left to stand at room temperature for 1 day. Then, the solution was hydrolyzed by addition of distilled water. The hydrolyzed material was extracted with ether, and the organic layer was distilled under reduced pressure, thus obtaining 2.3 g (65% yield) of 3-butylpyridazine (b.p.: 65-70° C., 0.5 mmHg) as oily liquid. Meanwhile, to 12 ml of anhydrous toluene, 3 mg (18 mM) of 2-bromopyridine and 15 mg (0.01 mM) of a palladium catalyst (pd(PPH₃)₄) were added and stirred for 5 minutes, and 130 mg (1 mM) of the above-prepared 3-butylpyridazine was then added. The mixture was allowed to react under reflux for 2 weeks. For complete reaction, 15 mg (0.01 mM) of pd(PPH₃)₄ was additionally added to the reaction mixture, followed by reaction for 2 weeks. The organic solvent was evaporated under reduced pressure, and the residue was purified by flash chromatography (20 g of Kieselgel 60; CH₂Cl₂: EtOAc=1:1), thus obtaining 82 mg (57% yield) of 2′-methylpyridazine as a semi-solid. The melting point and instrumental analysis results of the prepared compound are as follows.

M.p.: 87-89° C., 1H-NMR (250 MHz, CDCI3): δ 7.43 (ddd, IH, 3 J=4.8 Hz, 4 J=3.8 Hz, H5″), 7.84-7.92 (m, 2H, H3′/H4′), 8.07 (dd, IH, 4 J=2.4 Hz, 3 J=5.4 Hz, H5), 8.79 (ddd, IH, 3 J, 4 J=4.8 Hz, 5 J=1.3 Hz, H6′), 9.28 (dd, IH, 3 J=5.4 Hz, 5 J=1.3 Hz, H4), 9.80 (dd, IH, 4 J=2.4 Hz, 5 J=1.3 Hz, H6) ppm., Calcd. for C9H7N3(157.2): C, 68.77; H, 4.49; N 26.73. Found: C, 68.76; H, 4.69; N, 26.89., MS (EI-70 eV): 157(25) [M+], 156(100) [M+−H], 130(41) [M+−HCN], 79(74) [pyridyl+].

### PREPARATION EXAMPLE 7

### Preparation of 6′-methyl-2′-pyridylpyridazine

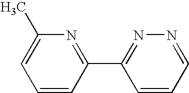

75 mg (53% yield) of 6′-methyl-2′-pyridylpyridazine was obtained in the same manner as in Preparation Example 6, except that 6-methyl-2-bromopyridine was used in place of 2-bromopyridine.

### TEST EXAMPLE 1

### Evaluation of Anticancer Activities of Inventive Pyridylpyridazine Compounds and Transition Metal Complexes Thereof

The pyridylpyridazine compounds prepared in Preparation Examples 1 to 5 and transition metal complexes thereof were evaluated for the anticancer activities thereof. As cancer cell lines used in the test, A549 (human lung cancer), SK-MEL-2 (human skin cancer), HCT-15 (human colon cancer), SK-OV-3 (human uterine cancer) and XF-498 (human brain cancer) among 60 kinds of cancer cell lines[Ross, D. T. et al., Systematic variation in gene expression patterns I human cancer cell lines. Nature genetics, 24, 227-235 (2000)], which are used for the search of anticancer activity in the US National Cancer Institute, were obtained from the Bioorganic Research Division, the Korea Research Institute of Chemical Technology. The anticancer activities of the cell lines were evaluated by measuring ED₅₀ values according to the sulforhodamine B (SRB) assay described in the literature[Choi, S. et al., Cytotoxicity of trichlorothecenes to human solid tumor cells in vitro. Arch. Pharm. Res. 19, 6-11 (1996)]. Also, to objectively determine the anticancer activities of the inventive compounds, cisplatin, which has been used as an anticancer agent in the prior art, was used as a positive control group.

More specifically, the SRB assay was performed in the following manner. 7 x cells/180 μl of each of the cancer cell lines were dispensed into each well of a 96-well plate. Each of the compounds prepared in Preparation Examples 1-5 was diluted with PBS and added to each well to concentrations of 0.1×10⁻⁸, 1×10⁻⁷, 1×10⁻⁶, 1×10⁻⁵, 1×10⁻⁴ and 1×10⁻³ M. Then, the cells were cultured for 48 hours. For use as a positive control group, cisplatin (Aldrich) was added to each of the cancer cell lines at the same concentrations as above. For use as a negative control group, the cell lines were not treated with anything. After completion of the culture process, the culture medium was removed from each well, and each of the cancer cell lines was immobilized with cold 10% trichloroacetic acid (TCA). To measure the death of each of the cancer cell lines, the cells were added to 0.4% SRB (sulforhodamine B) solution and then cultured at room temperature for 30 minutes. After completion of the culture, each well was well washed, treated with 150 μl (10 mM) of unbuffer ED Tris solution to dissolve out the SRB dye, and then measured for absorbance at 520 nm using a reader for 96-well plates. The average absorbance of each test group was expressed as percentage of the average absorbance of the wells (negative control group) untreated with the sample, and then a sample concentration, at which the average absorbance of the wells untreated with the sample could be reduced by 50%, i.e., 50% effective dose (ED₅₀), was calculated. The calculation results are shown in Table 1 below.

As can be seen from the test results, the inventive compound 3,6-bis(2′-pyridyl)pyridazine (compound of Preparation Example 1) showed low ED₅₀ for various human cancer cell lines, suggesting that it had high anticancer activity. Particularly, it showed high anticancer activity against the brain cell line. Also, the inventive compound 3,6-bis(6′-methyl-2′- pyridyl)pyridazine (compound of Preparation Example 2) showed low ED₅₀ for various human cancer cell lines, even though these ED50 values were higher than those of the compound of Preparation Example 1 (see Table 1). This suggests that the compound of Preparation Example 2 also had anticancer activity.

Meanwhile, the inventive copper complex of pyridylpyridazine (compound of Preparation Example 3) showed substantially the same level of low ED₅₀ values as that of cisplatin with respect to various human cancer cell lines except for the colon cancer cell line HCT-15, suggesting that it had very excellent anticancer activity. Also, the inventive zinc complex and nickel complex of pyridylpyridazine showed anticancer activity against various cancer cell lines, and particularly the zinc complex of pyridylpyridazine showed excellent anticancer activity against the brain cancer cell line.

TABLE 1

| | | SK- | | | |
|---|---|---|---|---|---|
| Compounds | A549¹⁾ | OV-3²⁾ | SK-MEL-2³⁾ | XF-498⁴⁾ | HCT-15⁵⁾ |
| Compound of | 5.24 | 4.77 | 6.05 | 1.88 | 3.79 |
| Preparation | | | | | |
| Example 1 | | | | | |
| Compound of | 12.26 | 17.80 | 13.08 | 16.44 | 16.02 |
| Preparation | | | | | |
| Example 2 | | | | | |
| Compound of | <1.25 | <1.25 | 2.36 | <1.25 | 25.27 |
| Preparation | | | | | |
| Example 3 | | | | | |
| Compound of | 17.39 | 21.07 | 14.88 | 17.25 | 5.26 |
| Preparation | | | | | |
| Example 4 | | | | | |
| Compound of | 29.73 | 17.24 | 26.47 | 18.84 | 20.42 |
| Preparation | | | | | |
| Example 5 | | | | | |
| Cisplatin | 1.24 | 0.83 | 0.79 | 0.88 | 2.13 |

Note:
¹⁾human lung cancer;
²⁾human uterine cancer;
³⁾human skin cancer,
⁴⁾human brain cancer, and
⁵⁾human colon cancer.

### TEST EXAMPLE 2

### Evaluation of Anticancer Activity of Copper Complex of Pyridylpyridazine Compound According to the Invention

The compound of Preparation Examples, which was found to show excellent anticancer activity in Test Example, that is, the copper complex of the pyridylpyridazine compound according to the present invention, was evaluated for anticancer activity against 12 kinds of human cancer cell lines. Five different melanoma cell lines, i.e., SK-MEL-5, SK-MEL-28, LOXIMVI, MALME-3M and M14, obtained from the Bioorganic Research Division, the Korea Research Institute of Chemical Technology, were used. As a colon cancer cell line, SW620 was used, and as an adenocarcinoma cell lines, DLD-1 was used. Also, as brain cancer cell lines, five different cell lines, i.e., SNB-19, SNB-75, SNB-78, U251 and SF298, were used. The anticancer activities of the compound of Preparation Example 3 and cisplatin against these cell lines were evaluated by measuring ED₅₀ values using the same method as described in Test Example 1. The evaluation results are shown in Table 2 below.

As can be seen from the test results in Table 2, the copper complex of the pyridylpyridazine compound according to the present invention showed substantially the same level of excellent anticancer activity as that of cisplatin against various cancer cell lines. Particularly, it showed excellent anticancer activity compared to that of cisplatin with respect to some cell lines, i.e., LDL-1 (adenocarcinoma), SK-MEL-28 (melanoma), SNB-19 (brain cancer), U251 (brain cancer) and SW620 (colon cancer) (see Table 2).

TABLE 2

| | Compound of Preparation | |
|---|---|---|
| Cancer cell lines | Example 3 | Cisplatin |
| DLD-11¹⁾ | 2.46 | 3.23 |
| SK-MEL-5²⁾ | 1.33 | 1.16 |
| SK-MEL-28²⁾ | 1.37 | 1.94 |
| LOXIMVI²⁾ | 1.00 | 0.23 |
| MALME-3M²⁾ | 3.28 | 1.06 |
| M14²⁾ | 1.48 | 1.19 |
| SNB-19³⁾ | 0.67 | 1.12 |
| SNB-75³⁾ | 2.83 | 1.20 |
| SNB-78³⁾ | 1.85 | 1.12 |
| U251³⁾ | 0.97 | 0.99 |
| SF-298³⁾ | 1.60 | 0.87 |
| SW620³⁾ | 1.74 | 3.26 |

Note:
¹⁾human adenocarcinoma;
²⁾human melanoma,
³⁾human brain cancer, and
⁴⁾human colon caner.

### INDUSTRIAL APPLICABILITY

As described above, the pyridylpyridazine compounds of Formula 1 according to the present invention and the transition metal complexes thereof have anticancer activity and can be easily prepared at low cost. Thus, these compounds are useful as anticancer agents against various cancers, including lung cancer, adenocarcinoma, skin cancer, colon cancer, uterine cancer and brain cancer, which are expressed due to immune system abnormality.

Although a few embodiments have been shown and described, it would be appreciated by those skilled in the art that changes may be made in these embodiments without departing from the principles and spirit of the invention, the scope of which is defined in the claims and their equivalents.

1. A pharmaceutical composition containing, as an active ingredient, a pyridylpyridazine compound of Formula 1 or a transition metal complex thereof: 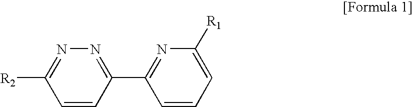
wherein R₁ is hydrogen or C₁—C₅ lower alkyl, R₂ is hydrogen or 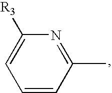
and R₃ is hydrogen or C₁—C₅ lower alkyl.

2. The pharmaceutical composition of claim 1, wherein the transition metal is selected from the group consisting of nickel, copper and zinc.

3. The pharmaceutical composition of claim 1, wherein the pyridylpyridazine compound is any one selected from the group consisting of 2′-pyridylpyridazine, 6′-methyl-2′-pyridylpyridazine, 3,6-bis(2′-pyridyl)pyridazine and 3,6-bis(6′-methyl-2′-pyridyl)pyridazine.

4. The pharmaceutical composition of claim 1, wherein the transition metal complex is any one selected from the group consisting of a [2′-pyridylpyridazine]zinc(II) complex, a [6′-methyl-2′-pyridylpyridazine]zinc(II) complex, a [3,6-bis(2′-pyridyl)pyridazine]zinc(II) complex, a [3,6-bis(6′-methyl-2′-pyridyl)pyridazine]zinc(II) complex, a bis[2′-pyridylpyridazine]copper(II) complex, a bis[6′-methyl-2′-pyridylpyridazine]copper(II) complex, a bis[3,6-bis(2′-pyridyl)pyridazine]copper(II) complex, a bis[3,6-bis(6′-methyl-2′-pyridyl)pyridazine]copper(II) complex, a [2′-pyridylpyridazine]nickel(II) complex, a [6′-methyl-2′-pyridylpyridazine]nickel(II) complex, a [3,6-bis(2′-pyridyl)pyridazine]nickel(II) complex and a [3,6-bis(6′-methyl-2′-pyridyl)pyridazine]nickel (II) complex.

5. The pharmaceutical composition of claim 4, wherein the transition metal is any one selected from the group consisting of bis[3,6-bis(2′ pyridyl)pyridazine]chlorocopper(II), bis[3,6-bis(6′-methyl-2′-pyridyl)pyridazine]chlorocopper(II) and bis[3 ,6-bis(6′-methyl-2′-pyridyl)pyridazine]chloronickel(II).
